(19) United States
(12) Patent Application Publication
(10) Pub. No.: US 2003/0073927 A1
(43) Pub. Date: Apr. 17, 2003

(54) Method for muting and/or un-muting of audio sources during a hearing test

(76) Inventors: Benny B. Johansen, Sunnyvale, CA (US); Ephram Cohen, San Francisco, CA (US); Sunil Puria, Mountain View, CA (US)

Correspondence Address: HAYNES BEFFEL & WOLFELD LLP, P O BOX 366, HALF MOON BAY, CA, 94019, US

(21) Appl. No.: 09/975,588

(22) Filed: Oct. 11, 2001

(51) Int. Cl. A61B 5/00; A61B 5/12

(52) U.S. Cl. 600/559; 73/585

(57) ABSTRACT

A method of testing the hearing of a user utilizing a computer system. The computer system includes a computer and a speaker. The computer includes a first audio source and a second audio source. In addition, the computer can output an electrical signal to the speaker from the first audio source and from the second audio source. Further, the speaker can convert the electrical signal into a stimulus. The method includes: downloading a computer program from a server to the computer; executing the computer program on the computer, the execution of the computer program muting the first audio source; generating a stimulus; and receiving an input from the user that indicates that the user heard the stimulus.

This patent application discloses subject matter that is related to the subject matter disclosed in U.S. patent application Ser. No. ______ entitled “Method For Setting Tone Controls During a Hearing Test,” Ser. No. ______ entitled “Method For Setting Volume and/or Balance Controls During a Hearing Test,” and Ser. No. ______ entitled “Method and System For Generating Audio Streams During a Hearing Test,” filed on even date herein. Each of the above patent applications is hereby incorporated by reference.

### FIELD OF THE INVENTION

The present invention generally relates to audiology and the presentation of audio signals for assessing a person's hearing using a personal computer. More specifically, the present invention relates to a system and a method for muting audio sources during a hearing test.

### BACKGROUND

Audiometric equipment exists for testing hearing. However, such equipment is expensive and is generally available only in hearing clinics. Many people are reluctant to visit hearing clinics and take a hearing test for a variety of reasons. Such reasons may include the cost of a hearing test, the time and inconvenience involved in scheduling of an appointment, waiting for and undergoing a hearing test, and privacy concerns. As a result, as many as 80 percent of the people who suffer from hearing loss in the United States may have not had their hearing tested.

Currently, a number of companies provide hearing tests over the Internet. For example, a user may have his/her hearing tested by accessing one of the following URLs: www.didyouhearme.com, www.handtronix.com, www.onlinehearing.com, www.audiainc.com, www.NigelWorks.com, w/w.audiologyawareness.com or www.freehearingtest.com.

For example, when a user accesses www.didyouhearme.com's hearing test, the user's computer system outputs a 500 Hz tone to a speaker, such as a powered or un-powered speaker or headphone that may include piezo electric transducers, which is coupled to the computer system. Next, the website instructs the user to decrease the volume on the user's computer until the user can no longer hear the 500 Hz tone.

Next, the website generates a 4000 Hz tone. According to the website, if the user cannot hear the 4000 Hz tone, the user may have a hearing impairment. Next, the website generates a number of tones at different amplitudes at the following frequencies: 250, 500, 750, 1000, 1500, 2000, 3000, and 4000 Hz. When the user hears a particular frequency, the user presses an acknowledge button on the screen with the computer's mouse. Based upon the user's acknowledgements, the website generates an audiogram. An audiogram is a chart plotting a user's hearing threshold level in dB HL as a function of frequency. Using the audiogram, the user can determine if the user has a hearing impairment.

Modem computer systems often have the ability to output sounds that are generated by a number of different audio sources. For example, many Windows operating systems include a Windows “Volume Control” applet window 100 such as shown in FIG. 1. This applet contains a number of volume controls 105, 110, 115, and 120. One volume control 105, a vertical slider bar in the “Wave” window of the applet, controls the amplitude of the electrical signals that are output to the user's computer system speakers when a user program sends a stream of digital amplitude values to the sound card driver. This stream may either be generated internally by the user's program or read from an auxiliary file, such as a wave file and routed to the sound card driver. Another volume control 110, a vertical slider bar in the “MIDI” window, controls the amplitude of the electrical signals that are output to the speakers when a user program sends a stream of midi data to the sound card data. The stream of midi data may be generated internally by the user's program, read from an auxiliary file, such as a midi file, or received from a connected MIDI source. Still another volume control 115, a vertical slider bar in the “CD Audio” window, controls the amplitude of the electrical signals that are output to the speakers if the audio source is a compact disk (“CD”). The Windows “Volume Control” applet may also contain other volume controls that control the output amplitude of electrical signals from other audio sources such as “Microphone,” “Synthesizer,” “Line-In,” “Auxiliary,” and “Modem.”

In addition to the above volume controls, the “Volume Control” applet window 100 also includes check boxes that mute individual audio sources. For example, the applet window 100 includes a check box 125 that mutes “Wave” audio sources, a check box 130 that mutes MIDI audio sources, and a check box 135 that mutes compact disk audio sources. In addition, a Windows “Volume control” applet window may also contain other check boxes that mute other audio sources such as “Microphone,” “Synthesizer,” “Line-In,” “Auxiliary” and “Modem.” In some implementations, the function of a “mute” control is inverted and the control is called “select”. In this case, if “select” is checked, the audio source will be un-muted, and if “select” is not checked, the audio source will be muted.

It is possible that noise from other audio sources could reduce the accuracy of the above-described hearing test. For example, if a microphone is coupled to the computer and the microphone input is not muted, then the microphone may detect the stimulus. Subsequently, the computer may amplify and output the detected stimulus through the speakers. At a minimum, the detected stimulus will decrease the accuracy of the hearing test. In addition, if the microphone volume level is set to a high setting, the detected stimulus may generate feedback, which would require halting the hearing test until the feedback is eliminated. In addition, other audio sources may decrease the accuracy of the hearing test by adding noise to the stimulus. Such noise may confuse the user taking the hearing test. Thus, a need exists for a hearing test that avoids such inaccuracies.

Further, some audio sources may be muted when a user starts the test. If a hearing test is designed to utilize such audio sources, these audio sources must be un-muted in order to produce an audible output and execute the hearing test. If these audio sources are muted, the test cannot be carried out unless the user is asked to manually change the settings in the Windows “Volume Control” applet window. This may be a difficult procedure for some computer users, and some people may not be able to take the hearing test, simply because they do not know how to un-mute certain audio sources.

### SUMMARY OF THE INVENTION

One embodiment of the invention is a method of testing the hearing of a user utilizing a computer system. The computer system includes a computer and a speaker. The computer includes a first audio source and a second audio source. In addition, the computer can output an electrical signal to the speaker from the first audio source and from the second audio source. Further, the speaker can convert the electrical signal into a stimulus. The method includes: downloading a computer program from a server to the computer; executing the computer program on the computer, the execution of the computer program muting and/or un-muting audio sources; generating a stimulus; and receiving an input from the user that indicates that the user heard the stimulus. In other embodiments of the invention, the method also includes: sending first data to the server; qualifying the hearing of the user; and sending second data to the computer.

Another embodiment of the invention is another method of testing the hearing of a user utilizing the above computer system. The method includes: downloading a computer program from a server to the computer; executing the computer program on the computer, the execution of the computer program storing a value that indicates whether the first audio source was muted and if the stored value indicates that the first audio source was not muted, then muting the first audio source; and/or storing a second value that indicates whether the second audio source was muted and if the second stored value indicates that the second audio source was muted, then un-muting the second audio source; generating a stimulus; receiving an input from the user that indicates that the user heard the stimulus; and, if the stored value indicates that the first audio source was not muted, then un-muting the first audio source; and if the second stored value indicates that the second audio source was muted then muting the second audio source. In other embodiments of the invention, the method also includes: sending first data to the server; qualifying the hearing of the user; and sending second data to the computer.

Still other embodiments of the invention include program storage devices that contain computer readable instructions that, when executed, perform portions of the above methods.

### BRIEF DESCRIPTION OF THE FIGURES

FIG. 1 presents a “Volume Control” applet window.

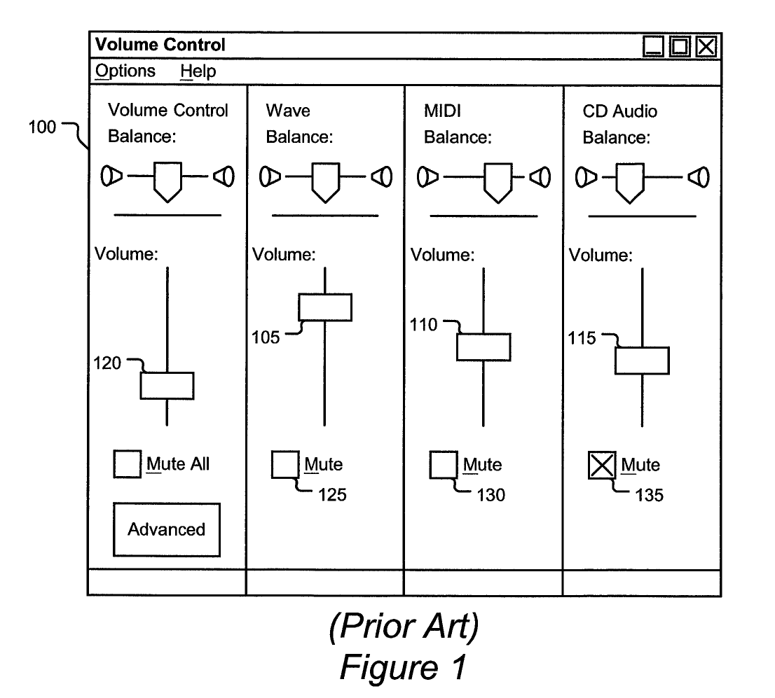

FIG. 2 presents a method of testing the hearing of a user that includes muting an audio source.

FIG. 3 presents another method of testing the hearing of a user that includes muting an audio source.

### DESCRIPTION OF THE PREFERRED EMBODIMENTS

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

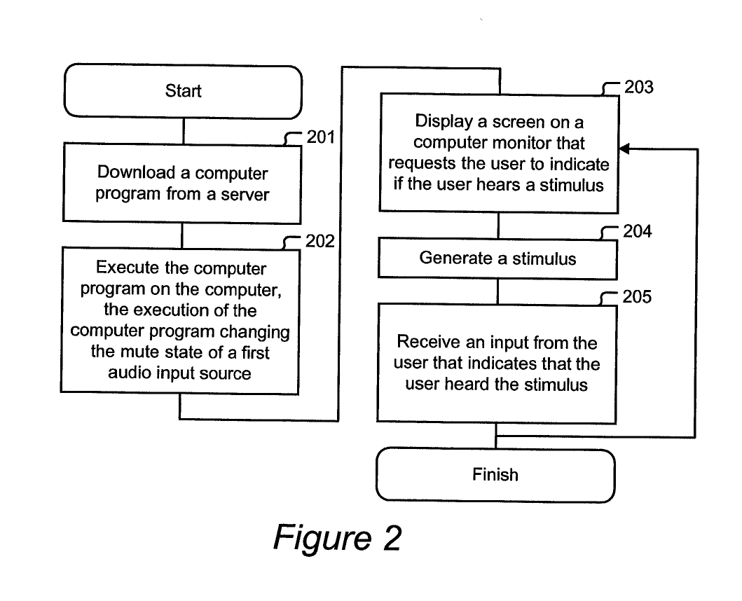

One embodiment of the invention, a method of testing the hearing of a user utilizing a computer system, is shown in FIG. 2. The method can be performed on a conventional computer system, such as a desktop computer system, a laptop computer system, or a handheld computer system. Other devices that include a microprocessor, such as a telephone, a mobile phone, a personal display assistant, an MP3 player, a radio, or a television, can also perform the method.

Hearing Tests that Modify the Mute State of an Audio Source

One embodiment of the invention, which is shown in FIG. 2, is a method of testing the hearing of a user utilizing a computer system. In this method, the computer program mutes one or more audio sources so that potential noise from these input audio sources is reduced. By eliminating such noise, the accuracy of a hearing test can be increased. Further, in some embodiments, the computer program un-mutes one or more other audio sources ensuring that audible sound will actually be generated from the sound card to the speaker.

Referring to Block 201 of FIG. 2, a user that desires to take a hearing test first downloads a computer program, such as a stand-alone executable program, a Java applet, an Active X control, or a Netscape plugin, from another computer, such as a server, to his/her computer. In one embodiment of the invention, the computer program is transferred via the Internet. In another embodiment of the invention, the computer program is transferred via an email. As is well known, computer programs may be attached to emails that can be easily distributed over the Internet, virtual private networks, local area networks and/or wide area networks. In still other embodiments, the computer program could be transferred to the user via the United States postal service or other postal service.

Next, referring to block 202 of FIG. 2, the user executes the computer program on the user's computer. When the computer program is executed, the computer program changes the mute state of one or more audio sources. For example, a microphone input, a line-in input, a CD input, a synthesizer input, an auxiliary input, and/or a modem input may be muted. Alternatively or additionally, the computer program may un-mute one or more audio sources. For example, the overall volume control, the MIDI control and/or the wave control could be un-muted.

Referring to block 203 of FIG. 2, the computer program then displays a screen on the computer monitor that requests the user to indicate if the user hears a stimulus. Then, referring to Block 204 of FIG. 2, the computer program generates a stimulus from another input audio source such as a Wave audio source. If the user hears the stimulus, then the user inputs information into the computer that indicates that the user hears the stimulus. Referring to Block 205 of FIG. 2, the computer program then receives the input from the user that indicates that the user heard the stimulus.

As shown in FIG. 2, by repeating Blocks 203 through 205 with stimuli of different amplitudes and frequencies, data sufficient to quantify the hearing of the user can be derived using conventional methods. In some embodiments of the invention, the computer program qualifies the hearing. In other embodiments, the computer program transfers data to a server and the server qualifies the hearing and then sends data back to the computer program. After the hearing of the user is quantified, some embodiments of the invention present an audiogram, text information, and/or graphical information to the user.

By muting one or more audio sources, the accuracy of the hearing test may be increased. For example, if a microphone is coupled to the computer and the microphone input is not muted, then the microphone may detect the stimulus. Subsequently, the computer may amplify the detected stimulus and output the detected stimulus through the speakers. At a minimum, the detected stimulus will decrease the accuracy of the hearing test. If the microphone volume level is set to a high setting, the detected stimulus may generate feedback, which would require halting the hearing test until the feedback is eliminated. Thus, muting one or more audio sources can increase the accuracy of the hearing test by eliminating noise in the stimulus. Such noise may confuse the user taking the hearing test.

Un-muting certain audio sources ensures that the signal generated by the program either directly or indirectly is indeed presented from the sound card to the speaker and to the user.

Other Embodiments of the Invention

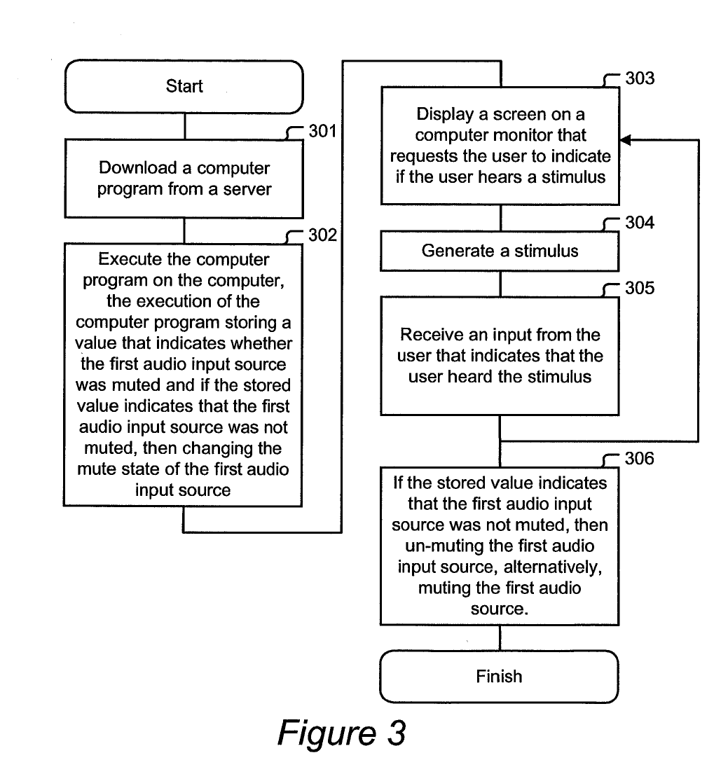

If the user had previously un-muted one or more audio sources, then the above method would “overwrite” such prior muting settings and mute the audio sources. Thus, in some embodiments of the invention, such as shown in FIG. 3, the computer program would store a value that indicates whether one or more audio sources was previously muted before muting the audio sources. Then, after the conclusion of the hearing test, the computer program would restore the mute state, i.e., muted or un-muted, of the audio sources before the initiation of the hearing test.

In other embodiments of the invention, if an audio source was previously muted, then the computer program would not attempt to mute the audio source. Alternatively, if certain audio sources were muted, the computer program would store a value that indicates the muted state of the audio source. Then, after the conclusion of the hearing test, the computer program would mute the audio sources, if they were muted before the initiation of the hearing test.

Conclusion

The foregoing descriptions of embodiments of the present invention have been presented for purposes of illustration and description only. They are not intended to be exhaustive or to limit the present invention to the forms disclosed. Accordingly, many modifications and variations will be apparent to practitioners skilled in the art. For example, program storage devices, such as hard disks, floppy disks, random access memories (RAM), read only memories (ROM), programmable read only memories (PROM), compact disks (CD), and digital versatile disks that contain computer readable instructions that perform portions of the above methods, are intended to be included in the present invention. Additionally, the above disclosure is not intended to limit the present invention. The scope of the present invention is defined by the appended claims.

It is claimed:

1. A method of testing the hearing of a user utilizing a computer system, the computer system including a computer and a speaker, the computer including a first audio source and a second audio source, the computer operable to output an electrical signal to the speaker from the first audio source and from the second audio source, the speaker operable to convert the electrical signal into a stimulus, the method comprising:
a) downloading a computer program from a server to the computer;
b) executing the computer program on the computer, the execution of the computer program muting the first audio source;
c) generating a stimulus; and
d) receiving an input from the user that indicates whether the user heard the stimulus.

2. The method of claim 1, wherein the act of downloading the computer program includes transferring the computer program from the server to the computer via the internet.

3. The method of claim 1, wherein the act of downloading the computer program includes transferring the computer program from the server to the computer via an email.

4. The method of claim 1, wherein the act of executing the computer program includes muting the first audio source by setting the value of a check box.

5. The method of claim 1, wherein the act of executing the computer program includes muting the first audio source by setting the value of a volume control.

6. The method of claim 1, wherein the act of executing the computer program includes muting the first audio source by setting the value of a check box and by setting the value of a volume control.

7. The method of claim 1, wherein the act of executing the computer program includes muting a microphone audio input.

8. The method of claim 1, further including:
a) sending first data to the server;
b) qualifying the hearing of the user; and
c) sending second data to the computer.

9. A method of testing the hearing of a user utilizing a computer system, the computer system including a computer and a speaker, the computer including a first audio source and a second audio source, the computer operable to output an electrical signal to the speaker from the first audio source and from the second audio source, the speaker operable to convert the electrical signal into a stimulus, the method comprising:
a) downloading a computer program from a server to the computer;
b) executing the computer program on the computer, the execution of the computer program storing a value that indicates whether the first audio source was muted and if the stored value indicates that the first audio source was not muted, then muting the first audio source;
c) generating a stimulus;
d) receiving an input from the user that indicates whether the user heard the stimulus; and
e) if the stored value indicates that the first audio source was not muted, then un-muting the first audio source.

10. The method of claim 9, wherein the act of downloading the computer program includes transferring the computer program from the server to the computer via the Internet.

11. The method of claim 9, wherein the act of downloading the computer program includes transferring the computer program from the server to the computer via an email.

12. The method of claim 9, wherein the act of executing the computer program includes muting the first audio source by setting the value of a check box.

13. The method of claim 9, wherein the act of executing the computer program includes muting the first audio source by setting the value of a volume control.

14. The method of claim 9, wherein the act of executing the computer program includes muting the first audio source by setting the value of a check box and by setting the value of a volume control.

15. The method of claim 9, wherein the act of executing the computer program includes muting a microphone audio input.

16. The method of claim 9, further including:
a) sending first data to the server;
b) qualifying the hearing of the user; and
c) sending second data to the computer.

17. A program storage device that contains computer readable instructions that, when executed by a computer system, tests the hearing of a user by:
a) muting an audio source;
b) generating a stimulus; and
c) receiving an input from the user that indicates whether the user heard the stimulus.

18. The program storage device of claim 17, wherein the act of muting the first audio source includes muting the first audio source by setting the value of a check box.

19. The program storage device of claim 17, wherein the act of muting the first audio source includes muting the first audio source by setting the value of a volume control.

20. The program storage device of claim 17, wherein the act of muting the first audio source includes muting the first audio source by setting the value of a check box and by setting the value of a volume control.

21. The program storage device of claim 17, wherein the act of muting the first audio source includes muting a microphone audio input.

22. A program storage device that contains computer readable instructions that, when executed by a computer system, tests the hearing of a user by:
a) storing a value that indicates whether a first audio source was muted;
b) if the first audio source was not muted, then muting the first audio source;
c) generating a stimulus;
d) receiving an input from the user that indicates whether the user heard the stimulus; and if the stored value indicates that the first audio source was not muted, then un-muting the first audio source.

23. The program storage device of claim 22, wherein the act of muting the first audio source includes muting the first audio source by setting the value of a check box.

24. The program storage device of claim 22, wherein the act of muting the first audio source includes muting the first audio source by setting the value of a volume control.

25. The program storage device of claim 22, wherein the act of muting the first audio source includes muting the first audio source by setting the value of a check box and by setting the value of a volume control.

26. The program storage device of claim 22, wherein the act of muting the first audio source includes muting a microphone audio input.

27. A method of testing the hearing of a user utilizing a computer system, the computer system including a computer and a speaker, the computer including a first audio source and a second audio source, the computer operable to output an electrical signal to the speaker from the first audio source and from the second audio source, the speaker operable to convert the electrical signal into a stimulus, the method comprising:
a) downloading a computer program from a server to the computer;
b) executing the computer program on the computer, the execution of the computer program un-muting the first audio source;
c) generating a stimulus; and
d) receiving an input from the user that indicates whether the user heard the stimulus.

28. The method of claim 27, wherein the act of downloading the computer program includes transferring the computer program from the server to the computer via the Internet.

29. The method of claim 27, wherein the act of downloading the computer program includes transferring the computer program from the server to the computer via an email.

30. The method of claim 27, wherein the act of executing the computer program includes un-muting the first audio source by setting the value of a check box.

31. The method of claim 27, wherein the act of executing the computer program includes un-muting the first audio source by setting the value of a volume control.

32. The method of claim 27, wherein the act of executing the computer program includes un-muting the first audio source by setting the value of a check box and by setting the value of a volume control.

33. The method of claim 27, wherein the act of executing the computer program includes un-muting a MIDI input.

34. The method of claim 27, wherein the act of executing the computer program includes un-muting a WAVE input.

35. The method of claim 27, further including:
a) sending first data to the server;
b) qualifying the hearing of the user; and
c) sending second data to the computer.

36. A program storage device that contains computer readable instructions that, when executed by a computer system, tests the hearing of a user by:
a) un-muting an audio source;
b) generating a stimulus; and
c) receiving an input from the user that indicates whether the user heard the stimulus.

37. The program storage device of claim 36, wherein the act of un-muting the first audio source includes un-muting the first audio source by setting the value of a check box.

38. The program storage device of claim 36, wherein the act of un-muting the first audio source includes un-muting the first audio source by setting the value of a volume control.

39. The program storage device of claim 36, wherein the act of un-muting the first audio source includes un-muting the first audio source by setting the value of a check box and by setting the value of a volume control.

40. The program storage device of claim 36, wherein the act of un-muting the first audio source includes un-muting a WAVE input.

41. The program storage device of claim 36, wherein the act of un-muting the first audio source includes un-muting a MIDI input.
